(19) United States
(12) Patent Application Publication
(10) Pub. No.: US 2006/0270714 A1
(43) Pub. Date: Nov. 30, 2006

(54) Novel n-substituted 2-aminopyridine derivatives

(76) Inventors: David Cheshire, Loughborough (GB); Stephen Connolly, Leicestershire (GB); Timothy Luker, Leicestershire (GB); Jeffrey Stonehouse, Leicestershire (GB)

Correspondence Address: FISH & RICHARDSON P.C., P.O BOX 1022, MINNEAPOLIS, MN, 55440-1022, US

(21) Appl. No.: 10/551,495

(22) PCT Filed: Mar. 30, 2004
(86) PCT No.: PCT/SE04/00488

§ 371 Date: Sep. 29, 2005

(30) Foreign Application Priority Data
Mar. 31, 2003 (SE) 0300906

(51) Int. Cl. C07D 213/78; A61K 31/44

(52) U.S. Cl. 514/352; 546/310

(57) ABSTRACT

There are provided novel compounds of formula (I) wherein R¹, R², R³, R⁴, R⁵, R⁶, R⁷, R⁸, R⁹, R¹⁷, L¹, L², L³ and Q are as defined in the specification, and pharmaceutically acceptable salts thereof; together with processes for their preparation, compositions containing them and their use in therapy. The compounds are inhibitors of nitric oxide synthase and are thereby particularly useful in the treatment or prophylaxis of inflammatory disease and pain.

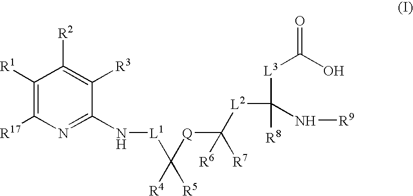

### FIELD OF THE INVENTION

The present invention relates to novel N-substituted 2-aminopyridine derivatives, processes for their preparation, compositions containing them and their use in therapy.

### BACKGROUND OF THE INVENTION

Nitric oxide is produced in mammalian cells from L-arginine by the action of specific nitric oxide synthases (NOSs). These enzymes fall into two distinct classes—constitutive NOS (cNOS) and inducible NOS (iNOS). At the present time, two constitutive NOSs and one inducible NOS have been identified. Of the constitutive NOSs, an endothelial enzyme (ecNOS) is involved with smooth muscle relaxation and the regulation of blood pressure and blood flow, whereas the neuronal enzyme (ncNOS) serves as a neurotransmitter and appears to be involved in the regulation of various biological functions such as cerebral ischaemia. Inducible NOS has been particularly implicated in the pathogenesis of inflammatory diseases. Regulation of these enzymes should therefore offer considerable potential in the treatment of a wide variety of disease states (J. E. Macdonald, Ann. Rep. Med Chem., 1996, 31, 221-230).

Considerable effort has been expended in efforts to identify compounds that act as specific inhibitors of one or more isoforms of the enzyme nitric oxide synthase. The use of such compounds in therapy has also been widely claimed.

### DISCLOSURE OF THE INVENTION

According to the present invention, there is provided a compound of formula (I)

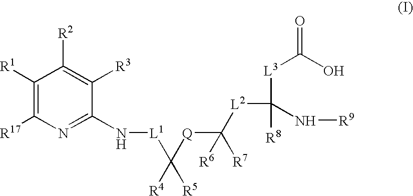

wherein

R¹, R², R³ and R¹⁷ independently represent H, halogen, C1 to 4 alkyl, C1 to 4 alkoxy, CN, MeS(O)ₘ or NR¹⁰R¹¹; said alkyl group being optionally further substituted by OH or one or more halogen atoms;

L¹represents CR¹²R¹³ wherein R¹² and R¹³ independently represent H or C1 to 4 alkyl; said alkyl being optionally further substituted by OH, C1 to 2 alkoxy, CN or one or more halogen atoms;

L² represents a bond or CR¹²R¹³ wherein R¹² and R¹³ independently represent H or C1 to 4 alkyl; said alkyl being optionally further substituted by OH, C1 to 2 alkoxy, CN or one or more halogen atoms;

L³ represents —CH₂— or a bond;

R⁴, R⁵, R⁶ and R⁷ independently represent H, C1 to 6 alkyl, Ar¹ or Ar¹-C1 to 4 alkyl;

or R⁴ and R⁵, or R⁶ and R⁷, may be joined together such that the group CR⁴R⁵ or the group CR⁶R⁷ represents a C3 to 6 cycloalkyl ring;

Q represents O, S(O)ₙ or NR¹⁶;

R¹⁶ represents H, C1 to 6 alkyl, C1 to 6 alkanoyl, C1 to 6 alkyl-SO₂—, C1 to 6 alkyl-O—CO—, Ar² or Ar²—CH₂—;

Ar¹ and Ar² independently represents phenyl or a 5- or 6-membered heteroaromatic ring containing one to three heteroatoms independently selected from O, S and N; said phenyl or heteroaromatic ring being optionally substituted by one or more substituents independently selected from halogen, CN, CF₃, C1 to 3 alkyl, C1 to 3 alkoxy, hydroxy, C1 to 3 thioalkoxy or NR¹⁴R¹⁵;

m and n independently represent an integer 0, 1 or 2;

R⁸ represents H or C1 to 4 alkyl; said alkyl being optionally further substituted by OH, C1 to 2 alkoxy, CN or one or more halogen atoms;

R⁹ represents H or C1 to 4 alkyl;

R¹⁰ and R¹¹ independently represent H, C1 to 2 alkyl, C1 to 2 alkanoyl or C1 to 2 alkylsulfonyl;

R¹⁴ and R¹⁵ independently represent H, C1 to 4 alkyl, C1 to 2 alkyl-SO₂—, or C1 to 4 alkanoyl; said alkyl being optionally further substituted by OH, C1 to 2 alkoxy, CN or one or more halogen atoms;

and pharmaceutically acceptable salts thereof.

Certain compounds of formula (I) are capable of existing in stereoisomeric forms. It will be understood that the invention encompasses all geometric and optical isomers of the compounds of formula (I) and mixtures thereof including racemates. Certain compounds of formula (I) are capable of existing in tautomeric forms. All such tautomers and mixtures thereof also form an aspect of the present invention.

In one embodiment, L³ represents a bond. In another embodiment, L¹ represents —CR¹²R¹³— wherein R¹² and R¹³ independently represent H or C1 to 4 alkyl. In another embodiment, L² represents a bond or —CR¹²R¹³ wherein R¹² and R¹³ independently represent H or C1 to 4 alkyl.

In one embodiment R² represents H, C1 to 4 alkyl or C1 to 4 alkoxy, and R¹ and R³ each represent H. In one embodiment, R² represents CH₃ or OCH₃.

In one embodiment, Q represents O.

In one embodiment, Q represents S.

In one embodiment, Q represents NR¹⁶ and R¹⁶ represents H or C1 to 6 alkyl.

In one embodiment, R⁴, R⁵, R⁶ and R⁷ each independently represent H or C1 to 4 alkyl.

The compounds of formula (I) and their pharmaceutically acceptable salts have the advantage that they are inhibitors of the enzyme nitric oxide synthase (NOS). In particular, the compounds of formula (I) and their pharmaceutically acceptable salts have the advantage that they are inhibitors of the inducible isoform of the enzyme nitric oxide synthase (iNOS).

The invention further provides a process for the preparation of compounds of formula (I) or a pharmaceutically acceptable salt, enantiomer or racemate thereof.

According to the invention there is also provided a compound of formula (I), or a pharmaceutically acceptable salt thereof for use as a medicament.

Another aspect of the invention provides the use of a compound of formula (I) or a pharmaceutically acceptable salt thereof, in the manufacture of a medicament, for the treatment or prophylaxis of diseases or conditions in which inhibition of nitric oxide synthase activity is beneficial.

A more particular aspect of the invention provides the use of a compound of formula (I) or a pharmaceutically acceptable salt thereof, in the manufacture of a medicament, for the treatment or prophylaxis of inflammatory disease.

According to the invention, there is also provided a method of treating, or reducing the risk of, diseases or conditions in which inhibition of nitric oxide synthase activity is beneficial which comprises administering to a person suffering from or at risk of, said disease or condition, a therapeutically effective amount of a compound of formula (I) or a pharmaceutically acceptable salt thereof.

More particularly, there is also provided a method of treating, or reducing the risk of, inflammatory disease in a person suffering from or at risk of, said disease, wherein the method comprises administering to the person a therapeutically effective amount of a compound of formula (I) or a pharmaceutically acceptable salt thereof.

The compounds of the present invention may also be used advantageously in combination with a second pharmaceutically active substance; particularly in combination with a cyclooxygenase inhibitor; more particularly in combination with a selective inhibitor of the inducible isoform of cyclooxygenase (COX-2). Thus, in a further aspect of the invention there is provided the use of a compound of formula (I) or a pharmaceutically acceptable salt thereof, in combination with a COX-2 inhibitor for the treatment of inflammation, inflammatory disease and inflammatory related disorders. And there is also provided a method of treating, or reducing the risk of, inflammation, inflammatory disease and inflammatory related disorders in a person suffering from or at risk of, said disease or condition, wherein the method comprises administering to the person a therapeutically effective amount of a compound of formula (I) or a pharmaceutically acceptable salt thereof in combination with a COX-2 inhibitor.

Particular compounds of the invention include:
S-[2-[(4-methyl-2-pyridinyl)amino]ethyl]-L-cysteine;
S-[2-[(4-methoxy-2-pyridinyl)amino]ethyl]-L-cysteine;
S-[2-[(4-methyl-2-pyridinyl)amino]pentyl]-L-cysteine;
S-[2-[(4-methyl-2-pyridinyl)amino]propyl]-L-cysteine;
   and pharmaceutically acceptable salts thereof.

Unless otherwise indicated, the term “C1 to 6 alkyl” referred to herein denotes a straight or branched chain alkyl group having from 1 to 6 carbon atoms. Examples of such groups include methyl, ethyl, n-propyl, i-propyl, n-butyl, i-butyl, t-butyl, pentyl and hexyl. The terms “C1 to 2 alkyl”, “C1 to 3 alkyl” and “C1 to 4 alkyl” are to be interpreted analogously.

Unless otherwise indicated, the term “C1 to 4 alkoxy” referred to herein denotes a straight or branched chain alkoxy group having from 1 to 4 carbon atoms. Examples of such groups include methoxy, ethoxy, n-propoxy and i-propoxy. The terms “C1 to 2 alkoxy” and “C1 to 3 alkoxy” are to be interpreted analogously.

Unless otherwise indicated, the term “C1 to 3 thioalkoxy” referred to herein denotes a straight or branched chain alkyl group having from 1 to 3 carbon atoms bonded to a sulphur atom. Examples of such groups include methylthio, ethylthio, n-propylthio and i-propylthio.

Unless otherwise indicated, the term “C3 to 6 cycloalkyl” referred to herein denotes a saturated carbocyclic ring having from 3 to 6 carbon atoms. Examples of such groups include cyclopropyl, cyclopentyl and cyclohexyl.

Unless otherwise indicated, the term “C1 to 6 alkanoyl” referred to herein denotes formyl or a straight or branched chain alkyl group having from 2 to 6 carbon atoms bonded to a carbonyl group. Examples of such groups include acetyl, n-propanoyl, i-propanoyl and butanoyl. The terms “C1 to 4 alkanoyl” and “C1 to 2 alkanoyl” are to be interpreted analogously.

Unless otherwise indicated, the term “halogen” referred to herein denotes fluoro, chloro, bromo and iodo.

Examples of a “C1 to 4 alkyl optionally further substituted by one or more halogen atoms” include CH₂F, CH₂Cl, CH₂Br, CHF₂, CF₃, CF₃CF₂, CF₃CH₂, CH₂FCH₂, CH₃CF₂ and CF₃CH₂CH₂.

Examples of a group “Ar¹-C1 to 4 alkyl” include Ar—CH₂—, Ar¹—CH₂CH₂— and Ar¹—CH(CH₃)—.

Examples of a 5- or 6-membered heteroaromatic ring containing one to three heteroatoms independently selected from O, S and N include furan, thiophene, thiazole, isoxazole, imidazole, triazole, thiadiazole, pyridine, pyrimidine and pyrazine.

Examples of a group “C1 to 6 alkyl-SO₂—” include methylsulphonyl, ethylsulphonyl and propylsulphonyl. The term “C1 to 2 alkylsulphonyl” denotes methylsulphonyl or ethylsulphonyl.

Examples of a group “C1 to 6 alkyl-O—CO—” include methoxycarbonyl, ethoxycarbonyl and propoxycarbonyl.

According to the invention, we further provide a process for the preparation of compounds of formula (I), or a pharmaceutically acceptable salt, enantiomer or racemate thereof which process [wherein variable groups are, unless otherwise specified, as defined in formula (I)] comprises:

(a) reaction of a compound of formula (II)

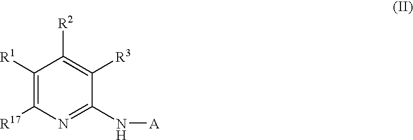

wherein A represents H, alkanoyl or carboxyalkanoyl,

with a compound of formula (III)

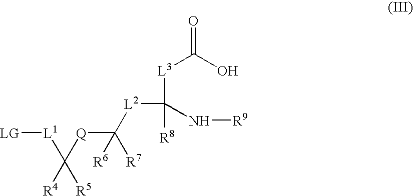

wherein LG represents a leaving group; or

(b) when Q represents S, reaction of a compound of formula (IV)

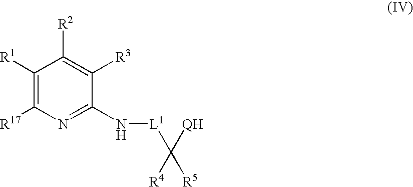

with a compound of formula (V)

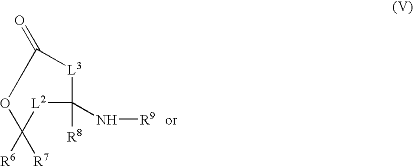

(c) when Q represents S, reacting a compound of formula (VI)

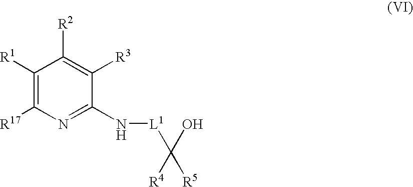

with a compound of formula (VII)

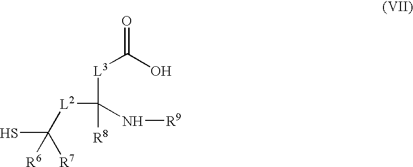

under Mitsunobu conditions;

and where desired or necessary converting the resultant compound of formula (I), or another salt thereof, into a pharmaceutically acceptable salt thereof; or converting one compound of formula (I) into another compound of formula (I); and where desired converting the resultant compound of formula (I) into an optical isomer thereof.

In processes (a) and (b), the reaction is performed by treating a nucleophile of formula (II) or (IV) with an electrophile of formula (III) or (V) respectively in an inert solvent. Suitable leaving groups LG include sulphonates and halides. The reaction is generally performed in the presence of a non-nucleophilic base such as sodium hydride, caesium carbonate, sodium bicarbonate or potassium hydroxide. Suitable organic solvents are those such as N,N-dimethylformamide, N-methyl-2-pyrrolidinone, tetrahydrofuran and dimethylsulfoxide. The reaction is generally conducted at a temperature between 0° C. and the boiling point of the solvent.

In process (c), the reactants (VI) and (VII) are coupled together in a suitable inert solvent such as tetrahydrofuran or dichloromethane using, for example, Mitsunobu conditions. Thus, for example, the reactants are treated with a phosphine derivative, an azo derivative and imidazole at a suitable temperature, generally between 0° C. and the boiling point of the solvent. Suitable phosphine derivatives include trimethylphosphine and tributylphosphine. Suitable azo derivatives include diethyl azodicarboxylate, diisopropyl azodicarboxylate, di-t-butyl azodicarboxylate and 1,1′-(azodicarbonyl)dipiperidine.

It will be apparent to a person skilled in the art that in the above processes it may be desirable or necessary to protect an amine, hydroxyl, carboxyl or other potentially reactive group. Suitable protecting groups and details of processes for adding and removing such groups may be found by reference to the standard text “Protective Groups in Organic Synthesis”, 3rd Edition (1999) by Greene and Wuts.

In one embodiment, amine groups are protected as carbamate derivatives, for example, as t-butyloxycarbamates. In another embodiment, carboxyl groups are protected as alkyl esters, for example, as methyl esters.

Specific examples of the use of protecting groups are given in the Examples section.

The present invention includes compounds of formula (I) in the form of salts, in particular acid addition salts. Suitable salts include those formed with both organic and inorganic acids. Such acid addition salts will normally be pharmaceutically acceptable although salts of non-pharmaceutically acceptable acids may be of utility in the preparation and purification of the compound in question. Thus, preferred salts include those formed from hydrochloric, hydrobromic, sulphuric, phosphoric, citric, tararic, lactic, pyruvic, acetic, succinic, fumaric, maleic, methanesulphonic and benzenesulphonic acids.

Salts of compounds of formula (I) may be formed by reacting the free base, or a salt, enantiomer or racemate thereof, with one or more equivalents of the appropriate acid. The reaction may be carried out in a solvent or medium in which the salt is insoluble or in a solvent in which the salt is soluble, for example, water, dioxane, ethanol, tetrahydrofuran or diethyl ether, or a mixture of solvents, which may be removed in vacuo or by freeze drying. The reaction may also be a metathetical process or it may be carried out on an ion exchange resin.

Intermediate compounds may be used as such or in protected form. Protecting groups and details of processes for their removal may be found by reference to the standard text “Protective Groups in Organic Synthesis”, 3rd Edition (1999) by Greene and Wuts.

The compounds of the invention and intermediates thereto may be isolated from their reaction mixtures and, if necessary further purified, by using standard techniques.

The compounds of formula I may exist in enantiomeric forms. Therefore, all enantiomers, diastereomers, racemates and mixtures thereof are included within the scope of the invention. The various optical isomers may be isolated by separation of a racemic mixture of the compounds using conventional techniques, for example, fractional crystallisation, or HPLC.

Intermediate compounds may also exist in enantiomeric forms and may be used as purified enantiomers, diastereomers, racemates or mixtures.

The compounds of formula (I), and their pharmaceutically acceptable salts are useful because they possess pharmacological activity in animals. In particular, the compounds are active as inhibitors of the enzyme nitric oxide synthase. More particularly, they are inhibitors of the inducible isoform of the enzyme nitric oxide synthase and as such are predicted to be useful in therapy, for example, as anti-inflammatory agents. They may also have utility as inhibitors of the neuronal isoform of the enzyme nitric oxide synthase.

The compounds and their pharmaceutically acceptable salts are indicated for use in the treatment or prophylaxis of diseases or conditions in which synthesis or oversynthesis of nitric oxide synthase forms a contributory part. In particular, the compounds are indicated for use in the treatment of inflammatory conditions in mammals including man.

Conditions that may be specifically mentioned are:

osteoarthritis, rheumatoid arthritis, rheumatoid spondylitis, gouty arthritic and other arthritic conditions, inflamed joints;

eczema, psoriasis, dermatitis or other inflammatory skin conditions such as sunburn;

inflammatory eye conditions including uveitis, glaucoma and conjunctivitis;

lung disorders in which inflammation is involved, for example, asthma, bronchitis, chronic obstructive pulmonary disease, pigeon fancier's disease, farmer's lung, acute respiratory distress syndrome;

bacteraemia, endotoxaemia (septic shock), aphthous ulcers, gingivitis, pyresis, pain, meningitis and pancreatitis;

conditions of the gastrointestinal tract including inflammatory bowel disease, Crohn's disease, atrophic gastritis, gastritis varialoforme, ulcerative colitis, coeliac disease, regional ileitis, peptic ulceration, irritable bowel syndrome, reflux oesophagitis, damage to the gastrointestinal tract resulting from infections by, for example, Helicobacter pylori, or from treatments with non-steroidal anti-inflammatory drugs;

and other conditions associated with inflammation.

The compounds will also be useful in the treatment and alleviation of acute pain or persistent inflammatory pain or neuropathic pain or pain of a central origin.

We are particularly interested in the conditions inflammatory bowel disease, rheumatoid arthritis, osteoarthritis, chronic obstructive pulmonary disease and pain.

The compounds of formula (I) and their pharmaceutically acceptable salts may also be useful in the treatment or prophylaxis of diseases or conditions in addition to those mentioned above. For example, the compounds may be useful in the treatment of atherosclerosis, cystic fibrosis, hypotension associated with septic and/or toxic shock, in the treatment of dysfunction of the immune system, as an adjuvant to short-term immunosuppression in organ transplant therapy, in the control of onset of diabetes, in the maintenance of pancreatic function in diabetes, in the treatment of vascular complications associated with diabetes and in co-therapy with cytokines, for example TNF or interleukins.

The compounds of formula (I) may also be useful in the treatment of hypoxia, for example in cases of cardiac arrest and stroke, neurodegenerative disorders including nerve degeneration and/or nerve necrosis in disorders such as ischaemia, hypoxia, hypoglycaemia, epilepsy, and in external wounds (such as spinal cord and head injury), hyperbaric oxygen convulsions and toxicity, dementia, for example pre-senile dementia, Alzheimer's disease and ADS-related dementia, Sydenham's chorea, Parkinson's disease, Tourette's syndrome, Huntington's disease, amyotrophic lateral sclerosis, multiple sclerosis, muscular dystrophy, Korsakoff's disease, imbecility relating to a cerebral vessel disorder, sleeping disorders, schizophrenia, depression, pain, autism, seasonal affective disorder, jet-lag, depression or other symptoms associated with premenstrual syndrome (PMS), anxiety and septic shock. Compounds of formula (I) may also be expected to show activity in the prevention and reversal of drug addiction or tolerance such as tolerance to opiates and diazepines, treatment of drug addiction, treatment of migraine and other vascular headaches, neurogenic inflammation, in the treatment of gastrointestinal motility disorders, cancer and in the induction of labour.

We are particularly interested in the conditions stroke, Alzheimer's disease, Parkinson's disease, multiple sclerosis, schizophrenia, migraine, cancer, septic shock and pain.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

For the above mentioned therapeutic indications, the dosage administered will, of course, vary with the compound employed, the mode of administration and the treatment desired. However, in general, satisfactory results are obtained when the compounds are administered at a dosage of the solid form of between 1 mg and 2000 mg per day.

The compounds of formula (I), and pharmaceutically acceptable derivatives thereof, may be used on their own, or in the form of appropriate pharmaceutical compositions in which the compound or derivative is in admixture with a pharmaceutically acceptable adjuvant, diluent or carrier. Administration may be by, but is not limited to, enteral (including oral, sublingual or rectal), intranasal, inhalation, intravenous, topical or other parenteral routes. Conventional procedures for the selection and preparation of suitable pharmaceutical formulations are described in, for example, “Pharmaceuticals—The Science of Dosage Form Designs”, M. E. Aulton, Churchill Livingstone, 1988. The pharmaceutical composition preferably comprises less than 80% and more preferably less than 50% of a compound of formula (I), or a pharmaceutically acceptable salt thereof.

According to the invention, we further provide a pharmaceutical composition comprising a compound of formula (I), or a pharmaceutically acceptable salt thereof, in admixture with a pharmaceutically acceptable adjuvant, diluent or carrier.

There is also provided a process for the preparation of such a pharmaceutical composition which comprises mixing the ingredients.

The compounds of formula (I), and pharmaceutically acceptable derivatives thereof, may also be advantageously used in combination with one of the following therapies: NSAIDS, COX-2 inhibitors, Paracetamol, Tramadol, Corticosteroids, Glucosamine, Doxycyclin, Pralnacasan, MMP inhibitors or Coll-3 inhibitors. The compound of formula (I) and the combination therapy may either be formulated together within the same pharmaceutical composition for administration in a single dosage unit, or each component may be individually formulated such that separate dosages may be administered either simultaneously or sequentially.

The invention is illustrated, but in no way limited, by the following examples:

The following abbreviations are used:

DMF N,N-Dimethylformamide;

THF Tetrahydrofuran;

DCM Dichloromethane.

Unless otherwise indicated, organic solutions were dried over anhydrous sodium sulphate.

### EXAMPLE 1

### S-[2-[(4-Methyl-2-pyridinyl)amino]ethyl]-L-cysteine acetate

a) 2-[(4-Methyl-2-pyridinyl)amino]-ethanol

2-Bromo-methyl-pyridine (3.6 ml) in 2-aminoethanol (10.8 ml) was heated at 160° C. for 16 h. The reaction mixture was cooled, dissolved in DCM and was washed with saturated potassium carbonate solution (3×) and dried (potassium carbonate). The solvent was evaporated to give the sub-title compound (4.3 g) as an oil.

MS APCI+ve^{m}/z 153 ([M+H]⁺).

¹H NMR 400 MHz (DMSO-d₆) 7.80 (1H, d), 6.31-6.28 (2H, m), 4.74 (1H, t), 3.52-3.47 (2H, m), 3.31-3.26 (2H, m), 2.13 (3H, s).

b) Benzenecarbothioic acid, S-[2-[(4-methyl-2-pyridinyl)amino]ethyl] ester

To a solution of triphenylphosphine (3.9 g) in THF (25 ml) under nitrogen at 0° C. was added diisopropylazodicarboxylate (2.9 ml) dropwise, the mixture was then stirred for 20 min. A solution of thiobenzoic acid (1.8 ml) and the product from step (a) (1.50 g) in THF (5 ml) was added dropwise. After the addition was complete the mixture was stirred at room temperature for 2 h. The mixture was concentrated and the residue purified by chromatography (silica, 50% diethyl ether/isohexane as eluent) to give the sub-title compound (0.70 g) as an orange oil.

MS APCI+ve^{m}/z 273 ([M+H ]⁺).

¹H NMR 400 MHz (CDCl₃) 8.11-7.39 (6H, m), 6.45 (1H, d), 6.40 (1H, s), 5.71 (1H, bs), 3.60 (2H, t), 3.33 (2H, t), 2.26 (3H, s).

c) S-[2-[(4-Methyl-2-pyridinyl)amino]ethyl]-L-cysteine acetate

The product from step (b) (0.70 g) in methanol (10 ml) was treated with 7M ammonia in methanol (20 ml) and stirred for 16 h. The solvent was evaporated to give an oil. This was taken up in the minimum of DMF and added dropwise to sodium hydride (0.10 g of a 60% dispersion in mineral oil) in DMF (10 ml) under nitrogen at 0° C. and stirred for 15 min. A solution of (S)-N-tert-butoxycarbonyl)-3-amino-2-oxetanone (0.37 g) in DMF (10 ml) was added dropwise to the mixture and stirred for 30 min. Additional (S)-N-(tert-butoxycarbonyl)-3-amino-2-oxetanone (0.11 g) in DMF (5 ml) was then added and stirred for a further 30 min. The reaction mixture was acidified with 10% aqueous potassium hydrogensulfate solution to pH 1-2 and the resulting white precipitate was filtered off. The filtrate was washed with ethyl acetate (2×), then DCM (2×). The aqueous mixture was evaporated and the residue purified by RPHPLC (symmetry column for stationary phase and 95-50 acetonitrile/ammonium acetate mobile phase). The relevant fractions were evaporated, then azeotroped with methanol and dried in vacuo to give the title compound as a white solid (10 mg).

MS APCI+ve^{m}/z 256 ([M+H]⁺).

¹H NMR 400 MHz (CD₃OD) 7.68 (1H, d), 6.34 (1H, d), 6.30 (1H, s), 3.64-3.60 (1H, m), 3.43-3.38 (2H, m), 3.10 (1H, dd), 2.90-2.84 (1H, m), 2.78-2.65 (2H, m), 2.13 (3H, s), 1.85 (3H, s).

### EXAMPLE 2

### S-[2-[(4Methoxy-2-pyridinyl)amino]ethyl]-L-cysteine

a) 2-[(4-Methoxy-2-pyridinyl)amino]-ethanol

Ethanolamine (4.0 ml) was added to potassium tert-butoxide (96 ml of a 1M solution in THF) and the reaction was stirred at room temperature for 30 min. 2-Chloro-4-methoxypyridine was added dropwise and the reaction mixture was heated under reflux for 16 h. The reaction mixture was cooled, filtered and evaporated to an oil. This was dissolved in xylene (100 ml) and treated with toluene-4-sulphonic acid (50 mg) and heated under reflux for 16 h. More toluene-4-sulphonic acid (50 mg) was added and the heating was continued for a further 16 h under reflux. The mixture was concentrated, the residue was passed through a pad of silica and eluted with 5% 7M ammonia in methanol/DCM to give the sub-title compound as a brown oil (5.0 g).

MS APCI+ve^{m}/z 169 ([M+H]⁺).

¹H NMR 300 MHz (DMSO-d₆) 7.76 (1H, d), 6.33 (1H, t), 6.13-6.10 (1H, m), 5.99 (1H, m), 3.70 (3H, s), 3.54-3.47 (2H, m), 3.31-3.25 (2H, m).

b) S-[2-[(4-Methoxy-2-pyridinyl)amino]ethyl]-L-cysteine

To a mixture of N-(tert-butoxycarbonyl)-L-cysteine methyl ester (2.12 g), the product from step (a) (0.50 g) and 1,1′-(azodicarbonyl)dipiperidine (1.51 g) in dry DCM (40 ml) under nitrogen was added imidazole (0.41 g). The reaction mixture was stirred for 5 min then trimethylphosphine (3 ml of a 1M solution in toluene), was added dropwise. After 4 h at room temperature, the reaction mixture was filtered, concentrated and the residue purified by chromatography (silica, 5% ammonia in methanol/DCM as eluent) to give an oil. This was then treated with 6M aqueous HCl (10 ml) and THF (1 ml) and heated at 100° C. for 3 h. The mixture was concentrated and the residue purified by RPHPLC (symmetry column for stationary phase and 95-50 acetonitrile/ammonium acetate mobile phase). The relevant fractions were evaporated, then azeotroped with methanol and the free amino acid was dried in vacuo to give the title compound as a white solid (9 mg).

MS APCI+ve^{m}/z 272 ([M+H ⁺).

¹H NMR 300 MHz (D₂O) 7.82 (1H, d), 6.45-6.42 (1H, m), 6.23-6.22 (1H, m), 3.97-3.89 (4H, m), 3.64-3.48 (2H, m), 3.18-3.03 (2H, m), 2.95-2.81 (2H, m).

### EXAMPLE 3

### S-[2-[(4-Methyl-2-pyridinyl)amino]pentyl]-L-cysteine dihydrochloride

a) 2-[(4-Methyl-2-pyridinyl)amino]pentan-1-ol

Potassium tert-butoxide (1.36 g) was added to a solution of 2-aminopentanol (1.24 g) in THF (20 ml) and stirred at room temperature for 20 min. 2-Chloro-4methylpyridine (0.9 ml) was added dropwise and the reaction stirred at room temperature for 90 min and heated at reflux for 3 h. The reaction was cooled and filtered, and the filtrate concentrated to give an oil which was dissolved in xylene (20 ml) and treated with a catalytic amount of toluene-4-sulphonic acid and heated at reflux for 24 h. The mixture was concentrated in vacuo, and the residue purified by chromatography (silica, 2% 7N NH₃ in methanol/DCM as eluent) to give the sub-title compound (1.08 g) as an off-white solid.

MS APCI+ve^{m}/z 195 ([M+H]⁺).

¹H NMR 300 MHz (CDCl₃) 7.87 (1H, d), 6.41 (1H, d), 6.27 (1H, s), 4.31 (1H, d), 3.87-3.77 (1H, m), 3.76 (1H, dd), 3.56 (1H, dd), 2.21 (3H, s), 1.65-1.33 (4H, m), 0.94 (3H, t).

b) S-[2-[(4-Methyl-2-pyridinyl)amino]pentyl]-L-cysteine dihydrochloride

A solution of product from step (a) (1.08 g), N-(tert-butoxycarbonyl)-L-cysteine methyl ester (4.08 g), 1,1′-(azodicarbonyl)dipiperidine (2.45 g) and imidazole (0.76 g) in dry, degassed DCM (50 ml) was stirred under nitrogen at room temperature for 10 min. Trimethylphosphine (11 ml of a 1M solution in toluene) was added dropwise and the reaction mixture was stirred for 1 h. The reaction mixture was then diluted with iso-hexane (60 ml), filtered through celite, concentrated and the residue purified by chromatography (silica, 0.5% NH₃ in methanol/DCM as eluent) to yield a yellow oil. This was then treated with 6N aqueous HCl (5 ml) and heated at reflux for 90 min. The reaction mixture was cooled, concentrated in vacuo, triturated in ether and dried in vacuo, to yield the title compound as yellow solid (48 mg).

MS APCI+ve^{m}/z 298 ([M+H]⁺).

¹H NMR 400 MHz (CD₃OD) 7.62 (1H, d), 6.82 (1H, s), 6.68 (1H, d), 4.11-4.16 (1H, m), 3.87-3.97 (1H, m), 3.00-3.16 (2H, m), 2.70-2.93 (2H, m), 2.31 (3H, s), 1.62-1.74 (1H, m), 1.48-1.60 (1H, m), 1.26-1.42 (2H, m), 0.87 (3H, t).

### EXAMPLE 4

### S-[2-[(Methyl-2-pyridinyl)amino]propyl]-L-cysteine ethanoate

a) 2-[(4-Methyl-2-pyridinyl)amino]propan-1-ol

2-Aminopropanol (0.49 ml) and 2-chloro-4-methylpyridine (0.45 ml) were reacted using the method described in Example 3 step (a) to give the sub-title compound (0.58 g) as an off-white solid.

MS APCI+ve^{m}/z 167 ([M+H]⁺).

¹H NMR 400 MHz (CDCl₃) 7.88 (1H, d), 6.43 (1H, d), 6.26 (1H, s), 4.29 (1H, s), 3.92-4.01 (1H, m), 3.73 (1H, dd), 3.55 (1H, dd), 2.22 (3H, s), 1.23 (3H, d).

b) S-[2-[(4-Methyl-2-pyridinyl)amino]propyl]-L-cysteine ethanoate

The product from step (a) (0.58 g) and N-(tert-butoxycarbonyl)-L-cysteine methyl ester (2.54 g) were reacted together using the method described in Example 3 step (b), and the residue purified by RPHPLC (symmetry column for stationary phase and 95-50 acetonitrile/ammonium acetate mobile phase). The relevant fractions were evaporated, then azeotroped with toluene and the product was dried in vacuo and the ethanoate salt formed to give the title compound as an off-white solid (19 mg).

MS APCI+ve^{m}/z 270 ([M+H]⁺).

¹H NMR 400 M (CD₃OD) 7.68 (1H, d), 6.33 (1H, d), 6.30 (1H, s), 3.97 (1H, quintet), 3.60-3.67 (1H, m), 2.70-3.17 (3H, m), 2.53-2.65 (1H, m), 2.12 (3H, s), 1.85 (3H, s), 1.18 (3H, d).

### Screens

The pharmacological activity of compounds according to the invention was tested in the following screens.

Screen 1

Recombinant human NO synthases (iNOS, eNOS & nNOS) were expressed in E. coli and lysates were prepared in Hepes buffer (pH 7.4) containing co-factors (FAD, FMN, H₄B), protease inhibitors, lysozyme and the detergent, CHAPS. These preparations were used, at suitable dilution, to assess inhibition of the various isoforms. Inhibition of NOS was determined by measuring the formation of L-[³H]citrulline from L-[³]arginine using an adaptation of the method of Förstermann et al.⁹ Enzyme assays were performed in the presence of 3 μM [³H]arginine, 1 mM NADPH and other co-factors required to support NOS activity (FAD, FMN, H₄B, calmodulin, Ca²⁺). Since various NOS inhibitors have been reported to exhibit slow binding kinetics, or to inactivate the enzyme in a time dependent manner, enzyme and inhibitor were pre-incubated for 60 min in the presence of NADPH before addition of arginine to initiate the reaction. Incubations continued for a further 60 min before the assays were quenched and [³H]citrulline separated from unreacted substrate by chromatography on Dowex-50W resin in a 96-well format.

In the above screen, the compounds of Examples 1 to 4 were tested and gave IC₅₀ values of less than 10 μM against the iNOS enzyme indicating that they are expected to show useful therapeutic activity.

Screen 2

Compounds also show activity against the human form of induced nitric oxide synthase as can be demonstrated in the following assay.

The human colorectal carcinoma cell line, DLD-1 (obtained from the European Collection of Animal Cell Culture—cell line number 90102540) was routinely grown in RPMI 1640 supplemented with 10% (v/v) foetal bovine serum, and 2 mM L-glutamine, at 37° C. in 5% CO₂.

Nitric oxide synthase was induced in cells by addition of medium containing human recombinant gamma-IFN (1000 units/ml), TNF-alpha (200 U/ml), IL-6 (200 U/ml) and

IL-1-beta (250 U/ml). After incubation for 18 hours at 37° C., the medium was removed and the cells washed with warm phosphate buffered saline. Cells were incubated for a further 5 hours at 37° C./5% CO₂ in RPMI 1640 containing 100 μM L-arginine and 100 μM verapamil-HCl in the presence and absence of test compounds.

Nitrite accumulation was determined by mixing an equal volume of culture media with Griess reagent (10 mg/ml sulphanilamide, 1 mg N-(1-naphthyl)ethylenediamine in 1 ml 2.5% (v/v) phosphoric acid). Inhibition in the presence of compounds was calculated relative to the nitrite levels produced by untreated cells. IC₅₀ values were estimated from a semi-log plot of % inhibition versus concentration of compound.

In this screen the compounds of Examples 1 to 4 gave IC₅₀ values of less than 100 μM, indicating that they are predicted to show useful therapeutic activity.

1. A compound of formula (I) 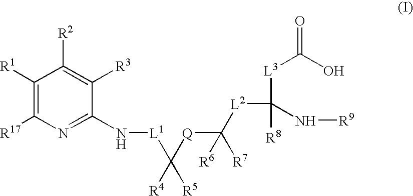
wherein
R¹, R², R³ and R¹⁷ independently represent H, halogen, C1 to 4 alkyl, C1 to 4 alkoxy, CN, MeS(O)ₘ or NR¹⁰R¹¹; said alkyl group being optionally further substituted by OH or one or more halogen atoms;
L¹ represents CR¹²R¹³ wherein R¹² and R¹³ independently represent H or C1 to 4 alkyl; said alkyl being optionally further substituted by OH, C1 to 2 alkoxy, CN or one or more halogen atoms;
L² represents a bond or CR¹²R¹³ wherein R¹² and R¹³ independently represent H or C1 to 4 alkyl; said alkyl being optionally further substituted by OH, C1 to 2 alkoxy, CN or one or more halogen atoms;
L³ represents —CH₂— or a bond;
R⁴, R⁵, R⁶ and R⁷ independently represent H, C1 to 6 alkyl, Ar¹ or Ar¹-C1 to 4 alkyl;
or R⁴ and R⁵, or R⁶ and R⁷, may be joined together such that the group CR⁴R⁵ or the group CR⁶R⁷ represents a C3 to 6 cycloalkyl ring;
Q represents O, S(O)ₙ or NR¹⁶;
R¹⁶ represents H, C1 to 6 alkyl, C1 to 6 alkanoyl, C1 to 6 alkyl-SO₂—, C1 to 6 alkyl-O—CO—, Ar² or Ar²—CH₂—;
Ar¹ and Ar² independently represents phenyl or a 5- or 6-membered heteroaromatic ring containing one to three heteroatoms independently selected from O, S and N; said phenyl or heteroaromatic ring being optionally substituted by one or more substituents independently selected from halogen, CN, CF₃, C1 to 3 alkyl, C1 to 3 alkoxy, hydroxy, C1 to 3 thioalkoxy or NR¹⁴R¹⁵;
m and n independently represent an integer 0, 1 or 2;
R⁸ represents H or C1 to 4 alkyl; said alkyl being optionally further substituted by OH, C1 to 2 alkoxy, CN or one or more halogen atoms;
R⁹ represents H or C1 to 4 alkyl;
R¹⁰ and R¹¹ independently represent H, C1 to 2 alkyl, C1 to 2 alkanoyl or C1 to 2 alkylsulfonyl;
R¹⁴ and R¹⁵ independently represent H, C1 to 4 alkyl, C1 to 2 alkyl-SO₂—, or C1 to 4 alkanoyl; said alkyl being optionally further substituted by OH, C1 to 2 alkoxy, CN or one or more halogen atoms;
and pharmaceutically acceptable salts thereof.

2. A compound according to claim 1 wherein Q represents S.

3. A compound of formula (I), according to claim 1, which is:
S-[2-[(4-methyl-2-pyridinyl)amino]ethyl]-L-cysteine;
S-[2-[(4-methoxy-2-pyridinyl)amino]ethyl]-L-cysteine;
S-[2-[(4-methyl-2-pyridinyl)amino]pentyl]-L-cysteine;
S-[2-[(4-methyl-2-pyridinyl)amino]propyl]-L-cysteine;
or a pharmaceutically acceptable salt thereof.

4. (canceled)

5. A pharmaceutical composition comprising a compound of formula (I) according to claim 1, or a pharmaceutically acceptable salt thereof, in admixture with a pharmaceutically acceptable adjuvant, diluent or carrier.

6-12. (canceled)

13. A method of treating, or reducing the risk of, human diseases or conditions in which inhibition of nitric oxide synthase activity is beneficial which comprises administering a therapeutically effective amount of a compound of formula (I), as defined in claim 1, or a pharmaceutically acceptable salt thereof, to a person suffering from, or at increased risk of, such diseases or conditions.

14. A method of treating, or reducing the risk of, inflammatory disease in a person suffering from, or at risk of, said disease, wherein the method comprises administering to the person a therapeutically effective amount of a compound of formula (I), as defined in claim 1, or a pharmaceutically acceptable salt thereof.

15. A process for the preparation of a first compound of formula (I), as defined in claim 1, or a pharmaceutically acceptable salt, enantiomer or racemate thereof, wherein the process comprises:
(a) reaction of a compound of formula (II) 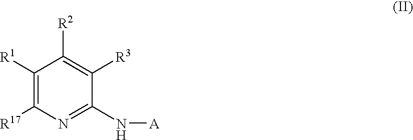
wherein A represents H, alkanoyl or carboxyalkanoyl,
with a compound of formula (III) 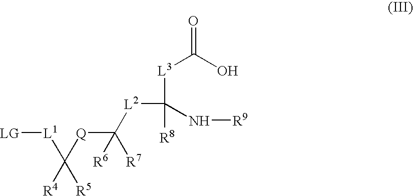
wherein LG represents a leaving group; or
(b) when Q represents S, reaction of a compound of formula (IV) 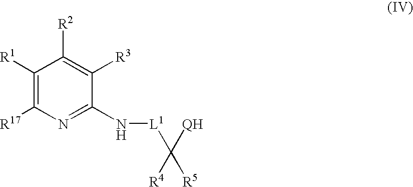
with a compound of formula (V) 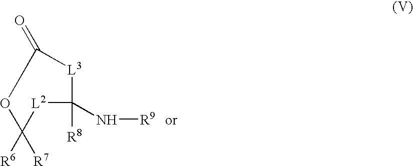
(c) when Q represents S, reacting a compound of formula (VI) 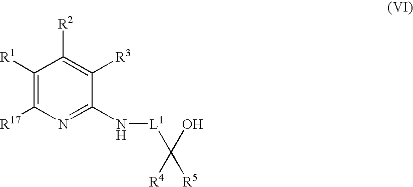
with a compound of formula (VII) 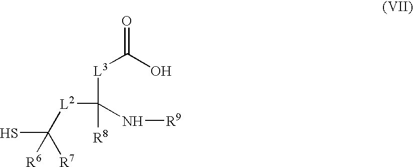
under Mitsunobu conditions;
under Mitsunobu conditions;
wherein the variable groups shown above are, unless otherwise specified, as defined in claim 1; and where desired or necessary converting the first compound of formula (I), or another salt thereof, into a pharmaceutically acceptable salt thereof; or converting the first compound of formula (I) into a second compound of formula (I); and where desired converting the first compound of formula (I) into an optical isomer thereof.

16. The method as claimed in claim 13, wherein it is predominantly inducible nitric oxide synthase that is inhibited.

17. The method as claimed in claim 14, wherein the disease is rheumatoid arthritis.

18. The method as claimed in claim 14, wherein the disease is osteoarthritis.

19. A method for the treatment or prophylaxis of pain, comprising administering a therapeutically effective amount of a compound of formula (I), as defined in claim 1, or a pharmaceutically acceptable salt thereof.

20. A method for the treatment or prophylaxis of inflammatory disease, comprising administering a therapeutically effective amount of a compound of formula (I) as defined in claim 1, or a pharmaceutically acceptable salt thereof, in combination with a COX-2 inhibitor.
